(19) United States
(12) Patent Application Publication
(10) Pub. No.: US 2010/0239063 A1
(43) Pub. Date: Sep. 23, 2010

(54) ROTATING ANODE X-RAY TUBE WIBH A SADDLE SHAPED ANODE

(75) Inventors: Rainer Pietig, Herzogenrath (DE); Astrid Lewalter, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/300,137

(22) PCT Filed: Apr. 26, 2007
(86) PCT No.: PCT/IB07/51561

§ 371 Date: Nov. 10, 2008

(30) Foreign Application Priority Data
May 8, 2006 (EP) 06113619.8

(51) Int. Cl. A61B 6/03; H01J 35/00

(52) U.S. Cl. 378/4; 378/126

(57) ABSTRACT

The rotating anode x-ray tube comprises an anode, whose focal track has the shape of a saddle trajectory. A proper anode angle to enable a high power line focus is always realised along the focal path on the anode. The x-ray tube is for CT scanners and enables a movement of the focal spot along the patient axis during gantry rotation. In this way, cone beam artefacts due to the reconstruction may be avoided.

The invention relates to the field of tomographic imaging. In particular, the invention relates to an examination apparatus for examination of an object of interest, to a method of examination of an object of interest, a computer-readable medium, a programme element, and to a rotating anode x-ray tube.

High power x-ray tubes are used in particular for computer tomography scanners. Here, the x-ray tube rotates together with the detector around the patient. In order to reconstruct volume images, the patient bed is moved slowly through the scanner such that the x-ray focal spot describes a helix around the patient. Modern scanners may have many detector slices and thus a large cone angle, which allows for fast movement of the patient bed.

However, the volume image reconstruction may not be exactly possible using such a helix trajectory. This is reflected in the fact that reconstruction artefacts increase with increasing cone angle. A further increase of the number of detector slices beyond a certain limit may thus not create any further benefit.

For computed tomography (CT) reconstruction, different kinds of focal trajectories may be used. If, for example, a trajectory is chosen, where the movement in z-direction is not linear but oscillating back and forth along the patient axis (z-axis), cone beam artefacts may be eliminated and exact volume image reconstruction may become possible.

The realisation of a focal path, which oscillates back and forth in z-direction, with a state of the art CT scanner and a rotating anode x-ray tube is difficult.

A simple saddle trajectory may be realised, if the tube moves two times back and forth during one gantry rotation, which may lead to oscillation frequencies in the order of 5-10 Hz for the z-axis movement. However, due to the large weight of the x-ray tube, this may not be feasible.

It would be desirable to provide for an improved generation of a focal spot trajectory enabling an exact image reconstruction in CT.

The invention provides an examination apparatus, a rotating anode x-ray tube, a method of examining an object of interest with an examination apparatus, a computer-readable medium and a programme element with the features according to the independent claims.

It should be noted that the following described exemplary embodiments of the invention apply also for the method of examination of an object of interest, for the computer-readable medium and for the programme element.

According to an exemplary embodiment of the present invention, an examination apparatus for examination of an object of interest may be provided, the examination apparatus comprising a rotating anode x-ray tube adopted for emitting an electromagnetic radiation beam with a focal spot to the object of interest, wherein the rotating anode x-ray tube comprises an anode with a focal track, and wherein the anode is adapted for rotating around a first rotation axis, such that the focal spot moves back and forth along a direction of a z-axis during rotation of the anode.

Therefore, the examination apparatus may be adapted for performing a reciprocating focal spot movement by simply rotating the anode of the x-ray tube. The reciprocating focal spot movement, which form depends on the focal track of the anode, may be such that an exact volume image reconstruction becomes possible without additionally moving the object of interest, which may be a patient.

Thus, the examination apparatus comprises a rotating anode x-ray tube which enables a movement of the focal spot along the patient axis during gantry rotation. In this way, cone-beam artefacts due to the reconstruction may be avoided.

According to another exemplary embodiment of the present invention, the focal track of the anode has a sinusoidal form, resulting in a sinusoidal back and forth movement of the focal spot.

According to another exemplary embodiment of the present invention, the focal track of the anode has an essentially triangular form, resulting in an essentially linear back and forth movement of the focal spot.

Therefore, the oscillation along the z-axis may have a different functional form than a sinusoidal form. The oscillation frequency may be an even number multiple of the anode rotation frequency. This may lead to a balanced mass distribution of the anode.

According to another exemplary embodiment of the present invention, the focal spot moves on a saddle trajectory during rotation of the anode.

Furthermore, according to another exemplary embodiment of the present invention, the x-ray tube is adapted for rotating around the z-axis around the object of interest.

Therefore, a movement of the focal spot along the patient axis is provided during gantry rotation.

According to another exemplary embodiment of the present invention, the examination apparatus further comprises a detector adapted for rotating together with the x-ray tube around the object of interest.

Furthermore, according to another exemplary embodiment of the present invention, the examination apparatus comprises a cathode adapted for emitting an electron beam towards the anode, and an electron optical lens system adapted for providing refocusing of the electron beam.

The electron optical lens system may be an advanced electron optical lens system.

The advanced electron optical lens system may be adapted to ensure, that the focus size remains unchanged while moving back and forth on the focal track. This is a challenging task in x-ray tube technology. In principle it may be done by a suitable combination of magnetic and/or electrostatic lenses.

According to another exemplary embodiment of the present invention, the first rotational axis of the anode is parallel to the z-axis, around which the x-ray tube rotates.

Furthermore, the anode may be further adapted for providing a small anode angle with respect to the electron beam, resulting in a high loadability. If the cone angle increases due to an increased number of detector rows, the anode angle may increase as well. Otherwise the entire cone may not be illuminated with x-rays.

In this context, high loadability means that the anode can withstand a high power electron beam, i.e. it can withstand a high thermal load. If the anode angle is increased, the loadability goes down, since the physical size of the focus on the anode becomes smaller, given that the projected focus size (as seen from the detector) remains.

According to another exemplary embodiment of the present invention, the examination apparatus is configured as one of the group consisting of a material testing apparatus, a medical application apparatus and a micro CT system.

A field of application of the invention may be medical imaging, in particular cardiac CT.

According to another exemplary embodiment of the present invention, the examination apparatus is adapted as one of a three dimensional computer tomography apparatus and a three dimensional rotational x-ray apparatus.

It should be noted in this context, that the present invention is not limited to computer tomography, but may always then be applied when the focal spot of an x-ray beam has to move in a reciprocating or oscillating manner.

According to another exemplary embodiment of the present invention, a rotating anode x-ray tube for an examination apparatus may be provided, the rotating anode x-ray tube adapted for emitting an electromagnetic radiation beam with a focal spot, the beam being emitted towards an object of interest to be examined, wherein the rotating anode x-ray tube comprises an anode with a focal track, wherein the anode is adapted for rotating around a first rotation axis such that the focal spot moves back and forth along a direction of a z-axis during rotation of the anode.

A proper anode angle to enable a high power line focus may always be realized along the focal path on the anode.

According to another exemplary embodiment of the present invention, the focal track of the anode has a sinusoidal or an essentially triangular form, resulting in a sinusoidal back and forth movement of the focal spot or in an essentially linear back and forth movement of the focal spot, respectively.

Furthermore, according to another exemplary embodiment of the present invention, a method of examination of an object of interest with an examination apparatus may be provided, the method comprising the step of emitting an electromagnetic radiation beam with a focal spot to the object of interest by a rotating anode x-ray tube, wherein the rotating anode x-ray tube comprises an anode with a focal track, wherein the anode is adapted for rotating around a first rotational axis, such that the focal spot moves back and forth along a direction of a z-axis during rotation of the anode.

This may provide for a focal trajectory generation enabling an exact image reconstruction without having to move the object of interest back and forth. Thus, for example, much higher oscillation frequencies may be achieved.

According to another exemplary embodiment of the present invention, a computer-readable medium may be provided, in which a computer programme of examination of an object of interest is stored which, when being executed by a processor, is adapted to carry out the above-mentioned method step.

Furthermore, according to another exemplary embodiment of the present invention, a programme element of examination of an object of interest is provided, which, when being executed by a processor, is adapted to carry out the above-mentioned method step.

It should be noted, that (at least for CT) the x-ray tube with the z-moving focus may also comprise a collimation unit (or aperture system) between the output window of the tube and the object of interest, and that this collimation unit is capable of adapting very fast to the different z-positions of the focus, such that for every position of the focus the entire object of interest can be projected onto the detector by means of the x-ray beam. This fast adaptable collimator could for example have moving parts, or could provide as many fixed collimation “slits” as there will be different focal positions along the z-axis.

The examination of the object of interest may be realised by the computer programme, i.e. by software, or by using one or more special electronic optimisation circuits, i.e. in hardware, or in hybrid form, i.e. by means of software components and hardware components.

It should be noted, that, if the system in question is a cone-beam CT, the computer programme may perform the reconstruction of images of the object of interest that are free of cone-beam artifacts—a feature that may be made possible by a special shape of the anode.

The programme element according to an exemplary embodiment of the present invention may preferably be loaded into working memories of a data processor. The data processor may thus be equipped to carry out exemplary embodiments of the methods of the present invention. The computer programme may be written in a suitable programming language, such as, for example, C++ and may be stored in a computer-readable medium, such as a CD-ROM. Also, the computer programme may be available from a network, such as the World Wide Web, from which it may be downloaded into image processing units of processors, or any suitable computers.

It may be seen as the gist of exemplary embodiment of the present invention that an examination apparatus is provided with a rotating anode x-ray tube having an anode whose focal track has such a shape, that the focal spot moves along the patient axis during rotation of the gantry. Therefore, a reciprocating movement of the patient table may no longer be necessary.

These and other aspects of the present invention will become apparent from and elucidated with reference to the embodiments described hereinafter.

Exemplary embodiments of the present invention will be described in the following, with reference to the following drawings.

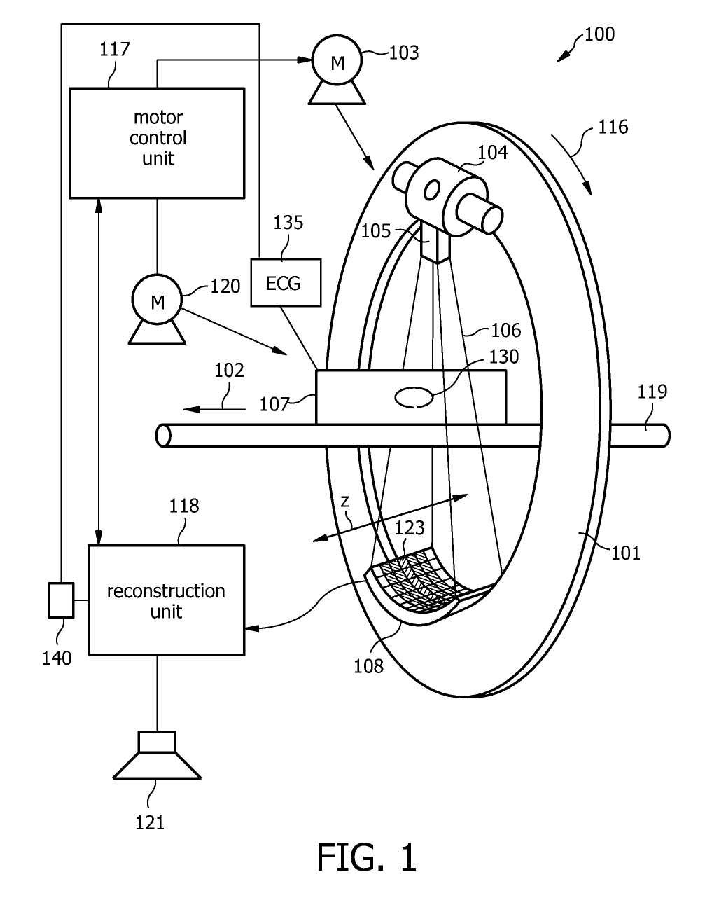

FIG. 1 shows a simplified schematic representation of an examination apparatus according to an exemplary embodiment of the present invention.

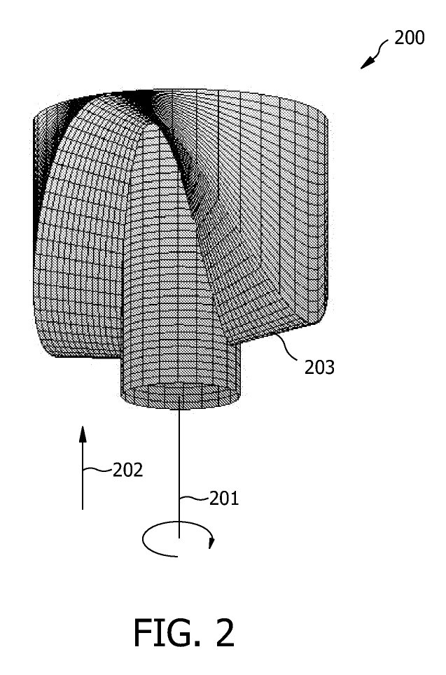

FIG. 2 shows a schematic representation of an anode according to an exemplary embodiment of the present invention.

FIG. 3 shows a modified saddle anode according to an exemplary embodiment of the invention.

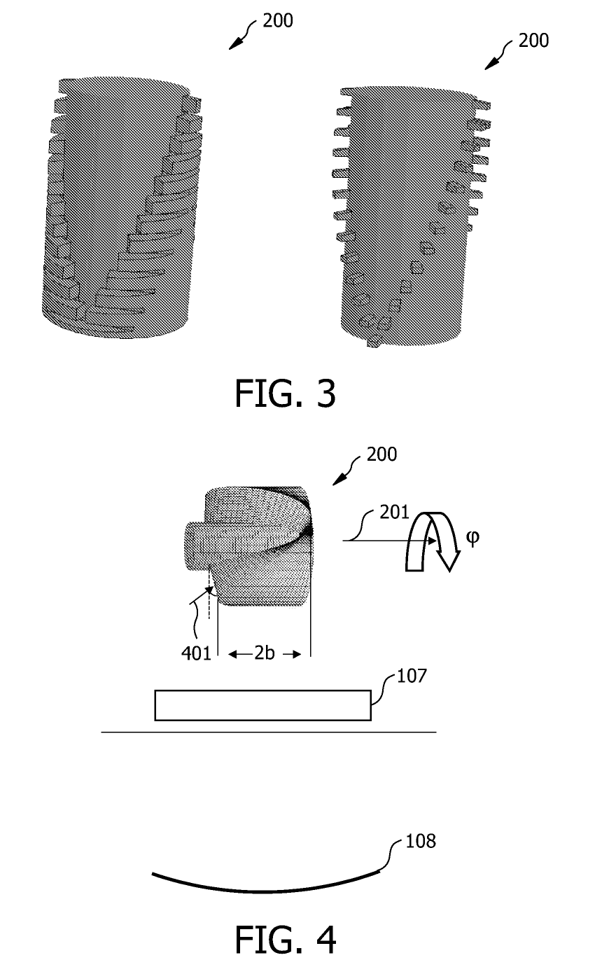

FIG. 4 shows a schematic representation of the geometrical setup according to an exemplary embodiment of the invention.

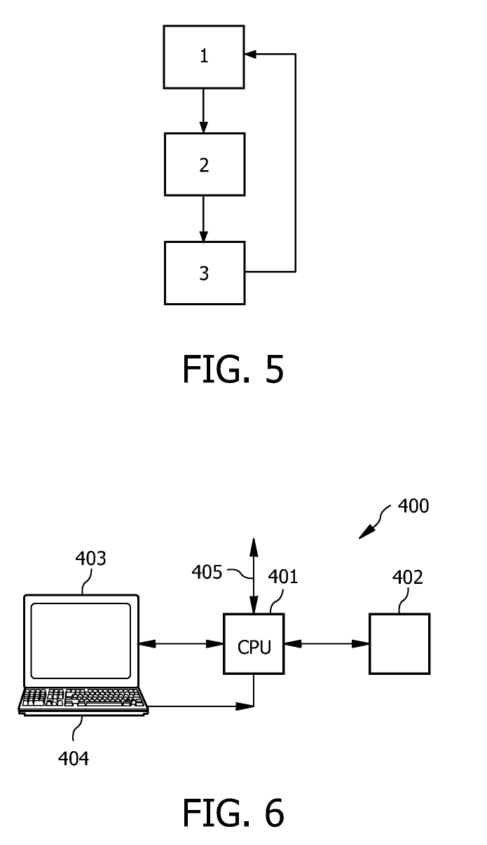

FIG. 5 shows a flow-chart of an exemplary method according to the present invention.

FIG. 6 shows an exemplary embodiment of an image processing device according to the present invention, for executing exemplary embodiment of a method in accordance with the present invention.

The illustration in the drawings is schematic. In different drawings, similar or identical elements are provided with the same reference.

FIG. 1 shows an exemplary embodiment of a computed tomography scanner system according to the present invention. The computer tomography apparatus 100 depicted in FIG. 1 is a cone-beam CT scanner. However, the invention may also be carried out with a fan-beam geometry. In order to generate a primary fan-beam, the aperture system 105 can be configured as a slit collimator. The CT scanner depicted in FIG. 1 comprises a gantry 101, which is rotatable around a rotational axis 102. The gantry 101 is driven by means of a motor 103. Reference numeral 104 designates a source of radiation such as an X-ray source, which, according to an aspect of the present invention, emits polychromatic or monochromatic radiation and comprises a rotating anode x-ray tube with a rotating anode 200 (see FIG. 2).

Reference numeral 105 designates an aperture system which forms the radiation beam emitted from the radiation source to a cone-shaped radiation beam 106. The cone-beam 106 is directed such that it penetrates an object of interest 107 arranged in the center of the gantry 101, i.e. in an examination region of the CT scanner, and impinges onto the detector 108. As may be taken from FIG. 1, the detector 108 is arranged on the gantry 101 opposite to the source of radiation 104, such that the surface of the detector 108 is covered by the cone beam 106. The detector 108 depicted in FIG. 1 comprises a plurality of detector elements 123 each capable of detecting X-rays which have been scattered by or passed through the object of interest 107.

During scanning the object of interest 107, the source of radiation 104, the aperture system 105 and the detector 108 are rotated along the gantry 101 in the direction indicated by an arrow 116. For rotation of the gantry 101 with the source of radiation 104, the aperture system 105 and the detector 108, the motor 103 is connected to a motor control unit 117, which is connected to a reconstruction unit 118 (which might also be denoted as a calculation or determination unit).

In FIG. 1, the object of interest 107 may be, for example, a human being which is disposed on an operation table 119. During the scan of, e.g., the heart 130 of the human being 107, while the gantry 101 rotates around the human being 107, the operation table 119 displaces the human being 107 along a direction parallel to the rotational axis 102 of the gantry 101. By this, the heart 130 is scanned along a helical scan path. The operation table 119 may also be stopped during the scans to thereby measure signal slices. It should be noted that in all of the described cases it is also possible to perform a circular scan, where there is no displacement in a direction parallel to the rotational axis 102, but only the rotation of the gantry 101 around the rotational axis 102.

Moreover, an electrocardiogram device 135 may be provided which measures an electrocardiogram of the heart 130 of the human being 107 while X-rays attenuated by passing the heart 130 are detected by detector 108. The data related to the measured electrocardiogram are transmitted to the reconstruction unit 118.

The detector 108 is connected to the control unit 118. The reconstruction unit 118 receives the detection result, i.e. the read-outs from the detector elements 123 of the detector 108 and determines a scanning result on the basis of these read-outs. Furthermore, the reconstruction unit 118 communicates with the motor control unit 117 in order to coordinate the movement of the gantry 101 with motors 103 and 120 with the operation table 119.

The reconstruction unit 118 may be adapted for reconstructing an image from read-outs of the detector 108. A reconstructed image generated by the reconstruction unit 118 may be output to a display (not shown in FIG. 1) via an interface 122.

The reconstruction unit 118 may be realized by a data processor to process read-outs from the detector elements 123 of the detector 108.

The computer tomography apparatus shown in FIG. 1 captures multi-cycle cardiac computer tomography data of the heart 130. In other words, when the gantry 101 rotates and when the operation table 119 is shifted linearly, then a helical scan is performed by the X-ray source 104 and the detector 108 with respect to the heart 130. During this helical scan, the heart 130 may beat a plurality of times. During these beats, a plurality of cardiac computer tomography data are acquired. Simultaneously, an electrocardiogram may be measured by the electrocardiogram unit 135. After having acquired these data, the data are transferred to the reconstruction unit 118, and the measured data may be analyzed retrospectively.

The measured data, namely the cardiac computer tomography data and the electrocardiogram data are processed by the reconstruction unit 118 which may be further controlled via a graphical user-interface (GUI) 140. This retrospective analysis is based on a helical cardiac cone beam reconstruction scheme using retrospective ECR gating.

It should be noted, however, that the present invention is not limited to this specific data acquisition and reconstruction.

FIG. 2 shows a schematic representation of an anode 200 according to an exemplary embodiment of the present invention. The anode 200 depicted in FIG. 2 comprises a focal track 203 which follows a saddle trajectory. The cathode, not depicted in FIG. 2, is located downward and emits an electron beam, which is represented by arrow 202. The electron beam 202 is oriented along the rotational axis 201 of the rotating anode 200.

As may be seen from FIG. 2, a small anode angle is realised everywhere along the focal path in order to ensure a high loadability.

The x-ray tube according to one embodiment of the present invention contains the rotating anode 200, whose focal path describes a saddle trajectory while the anode is rotating. The rotational axis 201 is parallel to the z-direction 102 of FIG. 1.

In addition, the x-ray tube contains a cathode, which emits an electron beam, and an advanced electron optical lens system, which may ensure the proper refocusing of the beam. The electron beam 202 is oriented parallel to the rotational axis 201.

The electron beam 202 follows the z-movement of the focal path accordingly while the anode is fast rotating around its axis 201.

The saddle shape may ensure that the focal spot is moving back and forth along the z-direction 102 while the anode 200 is rotating.

Furthermore, a small anode angle (as in normal rotating anode tubes) may be kept in order to ensure a high loadability.

The focal path of the anode according to an exemplary embodiment of the present invention may be parameterised as:

S(r,φ)=[r cos(α)cos(φ)r cos(φ)sin(φ)rsin(α)+b sin(2φ)]

Here, for fixed r and varying φ a saddle trajectory is described. For fixed φ and varying r the radial extension of the focal path is traced. α denotes the anode angle and 2b is the extension of the saddle trajectory along the rotational axis, as depicted in FIG. 4, and which may correspond to the rotational axis 102 of FIG. 1.

To maintain a high loadability, the focal track velocity should be as high as possible. This may enforce a fast movement of the focal spot in z-direction. E.g. for a rotation frequency of 100 Hz the distance 2b in z-direction is passed within 2.5 milliseconds, if the anode is shaped according to FIG. 2. The detector may have to be sufficiently fast to resolve the fast focal spot movement. This requirement may be relaxed for the “modified saddle anode” as described with respect to FIG. 3.

FIG. 3 shows modified saddle anodes according to exemplary embodiments of the invention. Here, the focal track contains discrete steps rather than a continuous path. Thus the focus position in z-direction is constant during a detector integration period.

As may be seen from FIG. 3, the oscillation along the z-axis may have a different functional form as given in the formula above. Instead of a sinusoidal form, an almost triangular form could be used (which is the case for the “modified saddle anode” of FIG. 3. Here, a triangular form may be beneficial since it may allow to avoid shadowing of the detector in fan direction, which may occur for the normal saddle anode between the extremal points of the saddle) to generate an almost linear back and forth movement of the focal spot.

The rotation of the anode is synchronized to the detector readout such that each integration period of the detector corresponds to one step (i.e. z-position) on the anode. In this way a smearing out of the focus along the z-direction for fast rotation frequencies may be avoided. Furthermore, a shadowing of the detector along the fan-angle may be avoided.

The oscillation frequency should be an even number multiple of the anode rotation frequency. Otherwise, the mass distribution of the anode may be unbalanced.

FIG. 4 shows a schematic representation of the geometrical setup according to an exemplary embodiment of the invention.

As described above, a denotes the anode angle and 2b is the extension of the saddle trajectory along the rotational axis 201, which may correspond to the rotational axis 102 (z-axis) of FIG. 1. The patient 107 is located between the detector 108 and the anode 200.

FIG. 5 shows a flow-chart of an exemplary method according to the present invention for examination of an object of interest. The method starts at Step 1 with the emission of an electron beam from a cathode towards an anode and then, in Step 2, the electron beam hits the rotating anode, thus generating an electromagnetic radiation beam with a focal spot, which is directed towards the object of interest.

Then, in Step 3, the anode is further rotated and the electron beam refocused by an electron optical lens system. Since the anode has further rotated and since the anode comprises a specifically formed focal track, the newly generated beam of electromagnetic radiation is now differently directed towards the object of interest and the aperture system needs to be adapted for this new beam position.

By repeating Steps 1, 2 and 3, a movement of the focal spot of the electromagnetic radiation beam is provided, which describes a saddle trajectory or any other trajectory, allowing an exact image reconstruction.

FIG. 6 depicts an exemplary embodiment of a data processing device 400 according to the present invention for executing an exemplary embodiment of a method in accordance with the present invention.

The data processing device 400 depicted in FIG. 6 comprises a central processing unit (CPU) or image processor 401 connected to a memory 402 for storing an image depicting an object of interest, such as a patient or an item of baggage. The data processor 401 may be connected to a plurality of input/output network or diagnosis devices, such as a CT device. The data processor 401 may furthermore be connected to a display device 403, for example, a computer monitor, for displaying information or an image computed or adapted in the data processor 401. An operator or user may interact with the data processor 401 via a keyboard 404 and/or other output devices, which are not depicted in FIG. 6.

Furthermore, via the bus system 405, it may also be possible to connect the image processing and control processor 401 to, for example, a motion monitor, which monitors a motion of the object of interest. In case, for example, a lung of a patient is imaged, the motion sensor may be an exhalation sensor. In case the heart is imaged, the motion sensor may be an electrocardiogram.

Exemplary embodiments of the invention may be sold as a software option to CT scanner console, imaging workstations or PACS workstations.

It should be noted that the term “comprising” does not exclude other elements or steps and the “a” or “an” does not exclude a plurality. Also elements described in association with different embodiments may be combined.

It should also be noted that reference signs in the claims shall not be construed as limiting the scope of the claims.

1. Examination apparatus (100) for examination of an object of interest (107), the examination apparatus (100) comprising:
a rotating anode x-ray tube (104) adapted for emitting an electromagnetic radiation beam with a focal spot to the object of interest (107);
wherein the rotating anode x-ray tube (104) comprises an anode (200) with a focal track; and
wherein the anode is adapted for rotating around a first rotational axis (201), such that the focal spot moves back and forth along a direction of a z-axis (102) during rotation of the anode.

2. The examination apparatus (100) of claim 1,
wherein the focal track of the anode has a sinusoidal form, resulting in a sinusoidal back and forth movement of the focal spot.

3. The examination apparatus (100) of claim 1,
wherein the focal track of the anode has an essentially triangular form, resulting in an essentially linear back and forth movement of the focal spot.

4. The examination apparatus (100) of claim 1,
wherein the focal spot moves on a saddle trajectory during rotation of the anode.

5. The examination apparatus (100) of claim 1,
wherein the x-ray tube (104) is adapted for rotating around the z-axis (102) around the object of interest (107).

6. The examination apparatus (100) of claim 1, the examination apparatus (100) further comprising:
a detector (108) adapted for rotating together with the x-ray tube (104) around the object of interest (107).

7. The examination apparatus (100) of claim 1, the examination apparatus (100) further comprising:
a cathode adapted for emitting an electron beam towards the anode; and
an electron optical lens system adapted for providing refocusing of the electron beam.

8. The examination apparatus (100) of claim 1,
wherein the first rotational axis (201) of the anode is parallel to the z-axis (102), around which the x-ray tube (104) rotates.

9. The examination apparatus (100) of claim 1,
wherein the anode is further adapted for providing a small anode angle with respect to the electron beam, resulting in a high loadability.

10. The examination apparatus (100) of claim 1, the examination apparatus (100) being configured as one of the group consisting of a material testing apparatus, a medical application apparatus and a micro CT system.

11. The examination apparatus (100) of claim 1,
wherein the examination apparatus (100) is adapted as one of a 3D computed tomography apparatus and a 3D rotational X-ray apparatus.

12. A rotating anode x-ray tube for an examination apparatus (100) and adapted for emitting an electromagnetic radiation beam with a focal spot to an object of interest (107) to be examined;
wherein the rotating anode x-ray tube (104) comprises an anode (200) with a focal track;
wherein the anode is adapted for rotating around a first rotational axis (201), such that the focal spot moves back and forth along a direction of a z-axis (102) during rotation of the anode.

13. The rotating anode x-ray tube of claim 12,
wherein the focal track of the anode has a sinusoidal form, resulting in an sinusoidal back and forth movement of the focal spot.

14. The rotating anode x-ray tube of claim 12,
wherein the focal track of the anode has an essentially triangular form, resulting in an essentially linear back and forth movement of the focal spot.

15. The rotating anode x-ray tube of claim 12,
wherein the focal spot moves on a saddle trajectory during rotation of the anode.

16. The rotating anode x-ray tube of claim 12,
wherein the x-ray tube (104) is adapted for rotating around the z-axis (102) around the object of interest (107).

17. A method of examination of an object of interest (107) with an examination apparatus (100), method comprising the steps of:
emitting an electromagnetic radiation beam with a focal spot to the object of interest (107) by a rotating anode x-ray tube (104);
wherein the rotating anode x-ray tube (104) comprises an anode (200) with a focal track;
wherein the anode is adapted for rotating around a first rotational axis (201), such that the focal spot moves back and forth along a direction of a z-axis (102) during rotation of the anode.

18. A computer-readable medium (402), in which a computer programme of examination of an object of interest is stored which, when being executed by a processor (401), is adapted to carry out the step of:
emitting an electromagnetic radiation beam with a focal spot to the object of interest (107) by a rotating anode x-ray tube (104);
wherein the rotating anode x-ray tube (104) comprises an anode (200) with a focal track;
wherein the anode is adapted for rotating around a first rotational axis (201), such that the focal spot moves back and forth along a direction of a z-axis (102) during rotation of the anode.

19. (canceled)
